(19) United States
(12) Patent Application Publication
(10) Pub. No.: US 2026/0076689 A1
(43) Pub. Date: Mar. 19, 2026

(54) MEDICAL DEVICE AND METHOD FOR MANUFACTURING MEDICAL DEVICE

(71) Applicant: ASAHI INTECC CO., LTD., Seto-shi (JP)

(72) Inventor: Issei ITO, Seto-shi (JP)

(73) Assignee: ASAHI INTECC CO., LTD., Seto-shi (JP)

(21) Appl. No.: 19/328,460

(22) Filed: Sep. 15, 2025

(30) Foreign Application Priority Data
Sep. 18, 2024 (JP) 2024-160689

(51) Int. Cl. A61B 17/22; A61B 17/00; A61M 25/09; C25F 3/16

(52) U.S. Cl. CPC A61B 17/22; A61M 25/09041; A61B 2017/00526; A61B 2017/22042; A61B 2017/22094; A61M 2025/09108; C25F 3/16

(57) ABSTRACT

A medical device, such as a guide wire, is to be inserted into a living body lumen such as a blood vessel. The medical device includes a main body part and a leading portion that is connected to a distal end of the main body part and enters a lesion, and the leading portion includes a protruding portion on a surface of the leading portion.

### CROSS REFERENCE TO RELATED APPLICATIONS

This is a Paris Convention application based on Japanese Patent Application JP 2024-160689. The disclosure of the prior application is hereby incorporated by reference herein in its entirety.

### TECHNICAL FIELD

The technology disclosed herein relates to a medical device and a method for manufacturing the medical device.

### BACKGROUND ART

Guide wires are used to treat stenosis sites or occlusion sites (hereinafter referred to as lesion) in blood vessels. Known guide wires include an elongate member. The elongate member includes a first portion having a first diameter and a second portion having a second diameter, and the second portion forms a loop (see, for example, Patent Document 1).

### CITATION LIST

Patent Document 1: Japanese Translation of PCT International Publication 2006-507899

### SUMMARY

### Technical Problem

Known guide wires have room for improvements in the passing performance with respect to a lesion.

This specification discloses a technology capable of solving the above-described issue.

### Solution to Problem

A medical device disclosed herein includes a main body part and a leading portion that is connected to a distal end of the main body part and enters a lesion, and the leading portion includes a plurality of protruding portions on a surface of the leading portion.

### BRIEF DESCRIPTION OF DRAWINGS

FIG. 1 is a plan view of a guide wire according to a first embodiment.

FIG. 2 is a side view of the guide wire according to the first embodiment.

FIG. 3 is a sectional view of the guide wire according to the first embodiment taken along the line III-III of FIG. 2.

FIG. 4 is a partially enlarged sectional view illustrating the inside of a frame F of FIG. 3 in an enlarged manner.

FIG. 5 is a partially enlarged sectional view illustrating the inside of a circle R1 of FIG. 4 in an enlarged manner.

FIG. 6 is a flowchart illustrating an example of a method for manufacturing the guide wire according to the first embodiment.

FIG. 7 is an explanatory view illustrating an example of the method for manufacturing the guide wire according to the first embodiment.

FIG. 8 is an explanatory view illustrating an example of the method for manufacturing the guide wire according to the first embodiment.

FIG. 9 is an explanatory view illustrating an example of the method for manufacturing the guide wire according to the first embodiment.

FIG. 10 is an explanatory view illustrating an example of a treatment method using the guide wire according to the first embodiment.

FIG. 11 is an explanatory view illustrating an example of the treatment method using the guide wire according to the first embodiment.

FIG. 12 is a partially enlarged sectional view illustrating a leading portion and a portion in the vicinity thereof of the guide wire according to a second embodiment, which is cut at the same position as the line III-III in FIG. 2.

FIG. 13 is a partially enlarged sectional view illustrating the inside of a circle R2 of FIG. 12 in an enlarged manner.

FIG. 14 is a flowchart illustrating an example of a method for manufacturing the guide wire according to the second embodiment.

FIG. 15 is a partially enlarged perspective view illustrating a leading portion and a portion in the vicinity thereof in a guide wire according to a third embodiment in an enlarged manner.

FIG. 16 is a partially enlarged sectional view of the guide wire according to the third embodiment, which is cut along the line XVI-XVI of FIG. 15.

FIG. 17 is a partially enlarged perspective view illustrating a leading portion and a portion in the vicinity thereof in a guide wire according to a fourth embodiment in an enlarged manner.

FIG. 18 is a partially enlarged perspective view illustrating a leading portion and a portion in the vicinity thereof in a guide wire according to a fifth embodiment in an enlarged manner.

### DESCRIPTION OF EMBODIMENTS

### First Embodiment

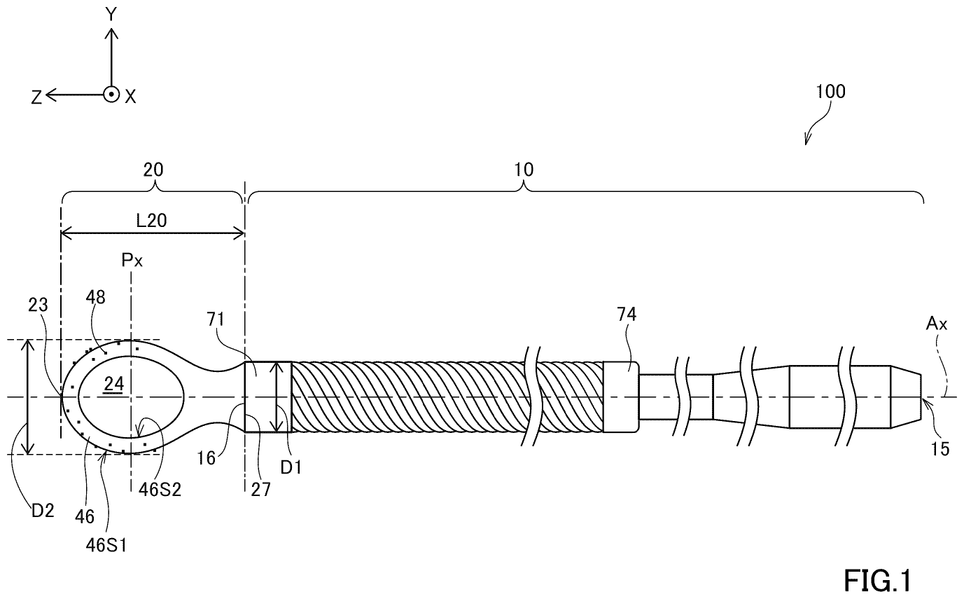

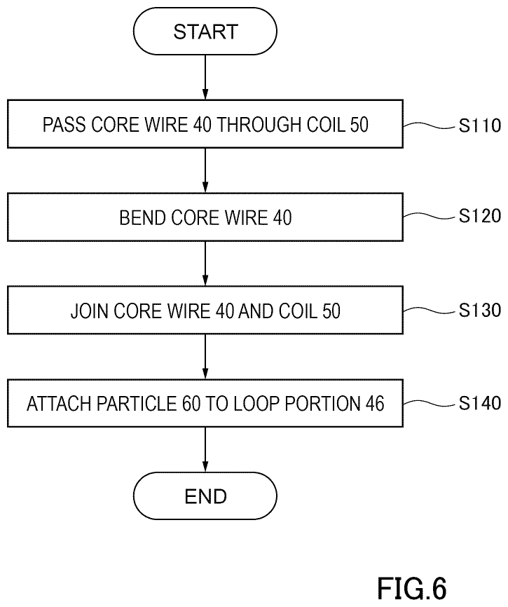

A first embodiment will be described with reference to FIGS. 1 to 11. A guide wire 100 according to the present embodiment is a medical device that is inserted into a living body lumen to treat a lesion in the living body lumen. The living body lumen includes a tubular organ of a human body such as a blood vessel, a digestive tract, a ureter, an organ, or a bile duct. In the guide wire 100, the Z-axis positive direction side is the distal end side to be inserted into a body, and the Z-axis negative direction side is a proximal end side to be operated by a professional such as a doctor. The distal end side is also referred to as a far side, and the proximal end side is also referred to as a near side. In each drawing, a part of the guide wire 100 may be omitted. FIGS. 1, 2 and 3 illustrate the guide wire 100 in a straight state parallel to the Z-axis. The guide wire 100 is flexible enough to be curved. The same applies to the subsequent drawings.

In this specification, with regard to the guide wire 100 and each constituent member thereof, an end on the distal end side is referred to as a “distal end”, the distal end and the vicinity thereof are referred to as a “distal end portion”, an end on the proximal end side is referred to as a “proximal end”, and the proximal end and the vicinity thereof are referred to as a “proximal end portion”. The transverse cross-section of the guide wire 100 and each constituent member thereof means a cross-section perpendicular to the longitudinal direction. The longitudinal cross-section of the guide wire 100 and each constituent component thereof means a cross-section parallel to the center axis in the longitudinal direction. In the guide wire 100 and each constituent component thereof, a direction perpendicular to the longitudinal direction is referred to as a radial direction. The outer diameter of the guide wire 100 and each constituent component thereof means a width in the radial direction.

The guide wire 100 is a medical device to be inserted into a living body lumen such as a blood vessel. The entire length of the guide wire 100 is, for example, 1000 mm or more and 3000 mm or less.

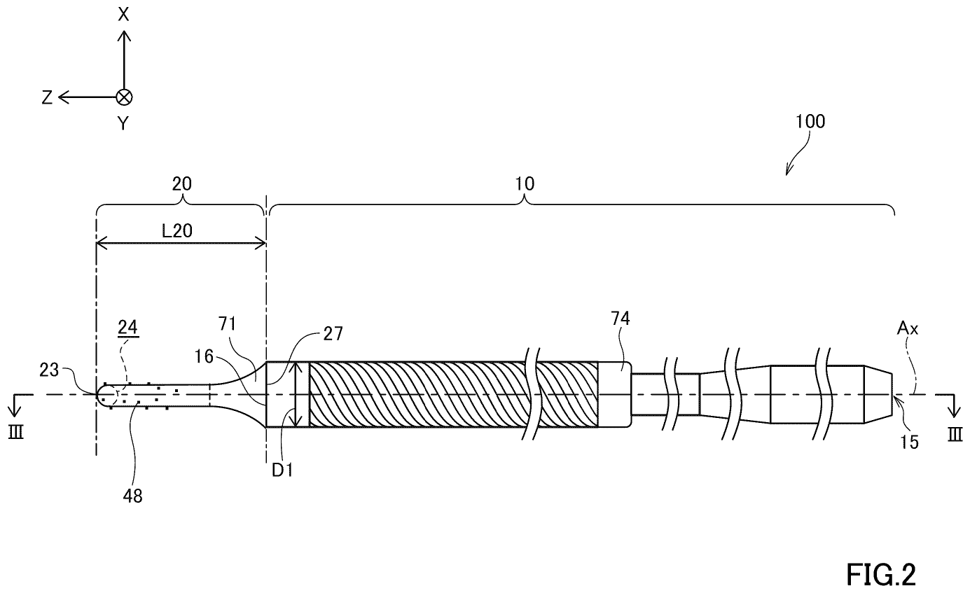

As illustrated in FIGS. 1 and 2, the guide wire 100 includes a main body part 10 and a leading portion 20.

The main body part 10 is an elongated portion extending along a center axis Ax. According to the present embodiment, the center axis Ax of the main body part 10 coincides with the center axis of the guide wire 100. A proximal end 15 of the main body part 10 coincides with the proximal end of the guide wire 100.

The leading portion 20 may be referred to as a lead portion, a drill portion, a crushing portion, a peeling portion, an entry portion, a peeler, a shaver, or the like. The leading portion 20 has a proximal end 27 connected to a distal end 16 of the main body part 10 and a distal end 23 opposite to the proximal end 27. The distal end 23 of the leading portion 20 coincides with the distal end of the guide wire 100. The surface of the leading portion 20 may have an edge or may have no edge. An edge is a boundary between two surfaces. The leading portion 20 enters a lesion while rotating around the center axis Ax. Entering the lesion by the leading portion 20 may be expressed as crossing or passing through the lesion, drilling the lesion, crushing the lesion, peeling the lesion, raking the lesion, going under the lesion, getting into the lesion, or the like. A length L20 of the leading portion 20 along the center axis Ax is, for example, 0.2 mm or more and 2.0 mm or less. The length L20 of the leading portion 20 may be 0.3 mm or more and 1.5 mm or less, or may be 0.4 mm or more and 1.0 mm or less.

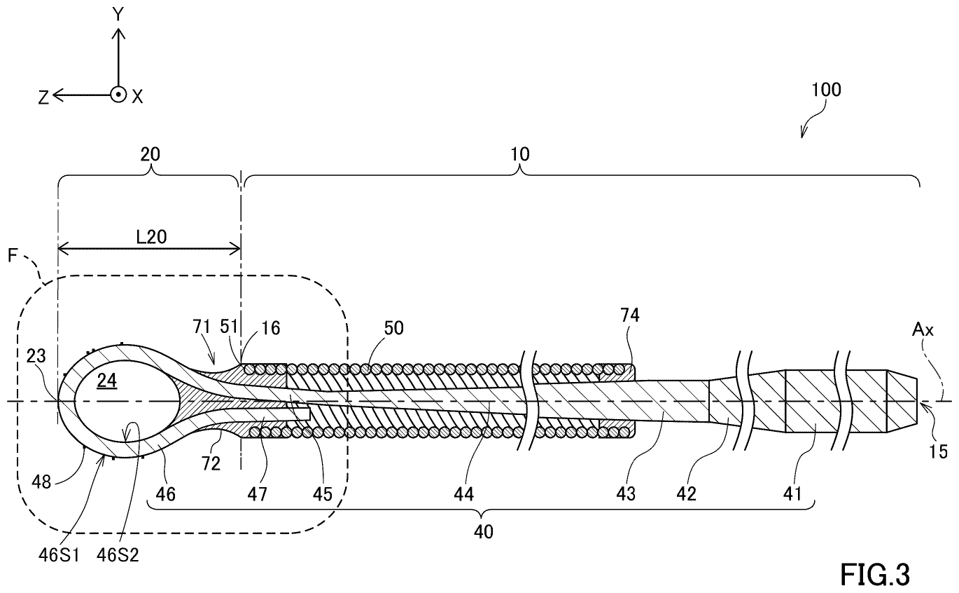

As illustrated in FIG. 3, the guide wire 100 includes a core wire 40 and a coil 50.

The coil 50 is a hollow cylindrical member in which one or more wire rods are spirally wound. According to the present embodiment, the coil 50 is a multi-thread coil formed by winding a plurality of wire rods. The outer diameter of the coil 50 is, for example, 0.1 mm or more and 0.6 mm or less. The outer diameter of the coil 50 may be 0.2 mm or more and 0.5 mm or less or may be 0.3 mm or more and 0.4 mm or less. The outer diameter of the coil 50 may be 1.00 mm or more and 2.00 mm or less, may be 1.10 mm or more and 1.65 mm or less, or may be 1.20 mm or more and 1.35 mm or less. According to the present embodiment, the outer diameter of the coil 50 is constant over the entire length of the coil 50. The coil 50 may have a tapered shape in which the outer diameter gradually decreases from the proximal end toward the distal end, or may have a tapered shape in which the outer diameter gradually decreases from the distal end toward the proximal end. A distal end 51 of the coil 50 substantially coincides with the distal end 16 of the main body part 10.

The wire rod forming the coil 50 may be a single strand or a twisted strand formed by twisting a plurality of strands. According to the present embodiment, the wire rod forming the coil 50 is a twisted strand.

The material of the coil 50 is, for example, metal. The material of the coil 50 may be a material that transmits radiation or may be a material that does not transmit radiation. The material that transmits radiation may be, for example, stainless steels such as SUS302, SUS304, and SUS316, Ni—Ti alloys, and piano wires. The material that does not transmit radiation may be, for example, platinum, gold, tungsten, or an alloy of any of these. The entire coil 50 may be formed of the same material, or each portion may be formed of a different material. The core wire 40 is a linear member. The core wire 40 includes a large diameter portion 41, a first tapered portion 42, an intermediate diameter portion 43, a second tapered portion 44, a first small diameter portion 45, a loop portion 46, and a second small diameter portion 47. The large diameter portion 41, the first tapered portion 42, the intermediate diameter portion 43, the second tapered portion 44, the first small diameter portion 45, the loop portion 46, and the second small diameter portion 47 are continuous in this order from the proximal end of the core wire 40. The loop portion 46 is an example of a base portion.

The large diameter portion 41 is a rod-shaped portion having a substantially constant outer diameter. The outer diameter of the large diameter portion 41 is, for example, about 0.2 mm to 3.0 mm. The intermediate diameter portion 43 is a rod-shaped portion that is located closer to the distal end side than the large diameter portion 41 and has a substantially constant outer diameter smaller than the outer diameter of the large diameter portion 41. The first tapered portion 42 is a portion that is located between the large diameter portion 41 and the intermediate diameter portion 43 and has a diameter gradually decreasing from the boundary with the large diameter portion 41 toward the boundary with the intermediate diameter portion 43. The first small diameter portion 45, the loop portion 46, and the second small diameter portion 47 are rod-shaped portions that are located closer to the distal end side than the intermediate diameter portion 43 and have a substantially constant outer diameter smaller than the outer diameter of the intermediate diameter portion 43. The second tapered portion 44 is a portion that is located between the intermediate diameter portion 43 and the first small diameter portion 45 and has a diameter gradually decreasing from the boundary with the intermediate diameter portion 43 toward the boundary with the first small diameter portion 45.

The second tapered portion 44, the first small diameter portion 45, and the second small diameter portion 47 of the core wire 40 are inserted into the inside of the coil 50.

The loop portion 46 is a portion of the core wire 40 between the first small diameter portion 45 and the second small diameter portion 47 and is located closer to the distal end side than the coil 50. The loop portion 46 is bent in a loop shape. More specifically, the loop portion 46 extends from the distal end of the first small diameter portion 45 toward the distal end while being curved, is folded back at the distal end, and extends toward the proximal end while being curved. In this specification, the term “loop shape” includes not only the case where the loop portion has only a closed ring, but also the case where the loop portion has a shape in which a part of the ring is cut out, and the case where a part of the loop portion extends to the outside of the ring. According to the present embodiment, most of the loop portion 46 except for both end portions forms a shape in which a part of an ellipse having a long axis in a direction along the center axis Ax is cut and both end portions extend substantially linearly toward the proximal end. According to the present embodiment, there is a clearance between both end portions of the loop portion 46. Both end portions of the loop portion 46 may be in contact with each other or may intersect each other. The shape of the ring formed by the loop portion 46 may be a circle, a partial circle, an ellipse, a rectangle, a parallelogram, a trapezoid, a rhombus, or the like, or may be a partially distorted shape of these shapes. The space inside the loop portion 46 is a through-hole 24 extending in the X-axis direction. The distal end of the loop portion 46 coincides with the distal end 23 of the leading portion 20.

The shape of the transverse cross-section of the core wire 40 at each position may be any shape. The shape of the transverse cross-section of the core wire 40 at each position may be a circle, a partial circle, an ellipse, a rectangle, a parallelogram, a trapezoid, a rhombus, or the like. A partial circle is one of the two halves of a circle divided by a chord. The outer edge of the partial circle includes an arc line and a line segment connecting two end portions of the arc line. The partial circle may have, for example, a semicircular shape, a partially circular shape, or an arcuate shape. A semicircular shape is one of two halves of a circle divided by a chord passing through the center of the circle. A partially circular shape is the larger of two halves of a circle divided by a chord that does not pass through the center of the circle. An arcuate shape is the smaller of the two halves divided by a chord that does not pass through the center of the circle. The transverse cross-section of the core wire 40 is not strictly limited to the above-described shapes, and may be substantially the above-described shapes. The shape of the transverse cross-section may be different at each position along the longitudinal direction of the core wire 40. According to the present embodiment, a portion of the core wire 40 serving as the loop portion 46 has a circular shape of the transverse cross-section. The surfaces of the loop portion 46 include an outer peripheral surface 46S1 and an inner peripheral surface 46S2. The inner peripheral surface 46S2 is a portion of the surface of the loop portion 46 that faces the internal space of the loop portion 46, and the outer peripheral surface 46S1 is the remaining portion of the surface of the loop portion 46 other than the inner peripheral surface 46S2.

The material of the core wire 40 is, for example, metal. To be more specific, the material of the core wire 40 may be, for example, stainless steels such as SUS302, SUS304, and SUS316, Ni—Ti alloys, and piano wires. The entire core wire 40 may be formed of the same material, or each portion of the core wire 40 may be formed of a different material.

The distal end portion of the coil 50 is joined to the core wire 40 by a distal end side joining member 71. A part of the distal end side joining member 71 enters the inside of the coil 50 and joins a part of the first small diameter portion 45, a part of the second small diameter portion 47, and the distal end portion of the coil 50. Another part of the distal end side joining member 71 serves as a reinforcing portion 72 protruding to the distal end side from the distal end 51 of the coil 50. The reinforcing portion 72 covers both end portions of the loop portion 46. The reinforcing portion 72 reinforces a connection portion of the leading portion 20 with the main body part 10. The proximal end portion of the coil 50 is joined to the core wire 40 with a proximal end side joining member 74. The coil 50 may be joined to the core wire 40 via a joining member formed at another position. The material of the distal end side joining member 71 and the proximal end side joining member 74 is, for example, solder, a brazing material, or an adhesive. The solder may be, for example, an Au—Sn alloy, an Sn—Ag alloy, an Sn—Pb alloy, or a Pb—Ag alloy. The brazing material may be, for example, an aluminum alloy brazing material, a silver brazing material, or a gold brazing material. The adhesive may be, for example, an epoxy-based adhesive.

The main body part 10 includes a portion of the core wire 40 from the large diameter portion 41 to the first small diameter portion 45, the second small diameter portion 47, the coil 50, a portion of the distal end side joining member 71 excluding the reinforcing portion 72, and the proximal end side joining member 74. The leading portion 20 includes the loop portion 46 formed by a part of the core wire 40 and the reinforcing portion 72.

As illustrated in FIG. 1, the outer diameter of the leading portion 20 changes along the center axis Ax as viewed in the X-axis direction. Specifically, the outer diameter of the leading portion 20 at the proximal end 27 of the leading portion 20 is substantially the same as a maximum outer diameter D1 of the distal end 16 of the main body part 10. The outer diameter of the leading portion 20 gradually decreases from the proximal end 27 of the leading portion 20 toward the distal end side, then gradually increases to reach a maximum outer diameter D2 at a maximum outer diameter position Px, and gradually decreases from the maximum outer diameter position Px to the distal end 23 of the leading portion 20. The maximum outer diameter D2 of the leading portion 20 is, for example, 0.2 mm or more and 1.0 mm or less. The maximum outer diameter D2 of the leading portion 20 may be 0.3 mm or more and 0.8 mm or less, or may be 0.4 mm or more and 0.6 mm or less. The maximum outer diameter D2 of the leading portion 20 may be 1.00 mm or more and 3.00 mm or less, may be 1.20 mm or more and 2.50 mm or less, or may be 1.50 mm or more and 2.00 mm or less.

The maximum outer diameter D2 of the leading portion 20 is measured as follows. The measurer observes the guide wire 100 from the side. The side is in the Y-axis direction according to the present embodiment. The measurer searches for the angle at which the near side portion and the far side portion of the loop portion 46 overlap each other and the far side portion is invisible. For example, when both of the following two conditions are satisfied, the measurer searches for the angle at which the portion between the second small diameter portion 47 and the distal end 23 is invisible. The first condition is that the portion between the first small diameter portion 45 and the distal end 23 is on the near side. The second condition is that the portion between the second small diameter portion 47 and the distal end 23 is on the far side. The non-visibility of the portion between the second small diameter portion 47 and the distal end 23 is caused by the overlap between the portion between the first small diameter portion 45 and the distal end 23 and the portion between the second small diameter portion 47 and the distal end 23. Subsequently, the measurer captures the guide wire 100 using a microscope along the viewpoint rotated by 90 degrees around the center axis Ax from the viewpoint at this time. The measurer sets the capture magnification of the microscope to 200 times or more. The measurer measures the outer diameter of the leading portion 20 at three measurement positions where the leading portion 20 is considered to have the maximum outer diameter D2 on the captured image. To be specific, at each measurement position, the measurer draws a pair of straight lines that pass through a pair of end portions of the leading portion 20 in the outer diameter direction, are perpendicular to the outer diameter direction, and are parallel to each other and measures the interval between the pair of straight lines. The measurer adopts the maximum value of the measurement results at the three measurement positions as the maximum outer diameter D2 of the leading portion 20.

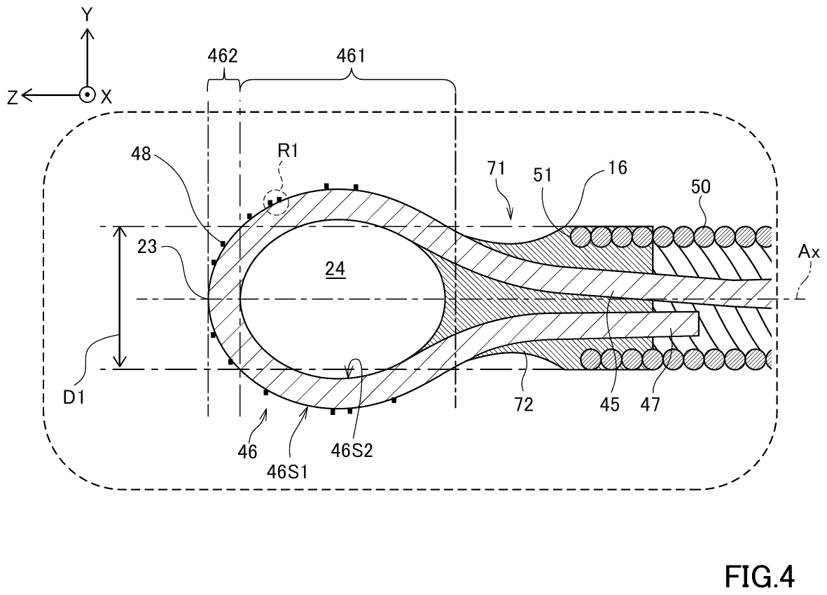

As illustrated in FIG. 4, the loop portion 46 includes a wide portion 461 having an outer diameter larger than the maximum outer diameter D1 of the distal end 16 of the main body part 10 and a distal end portion 462 located closer to the distal end side than the wide portion 461.

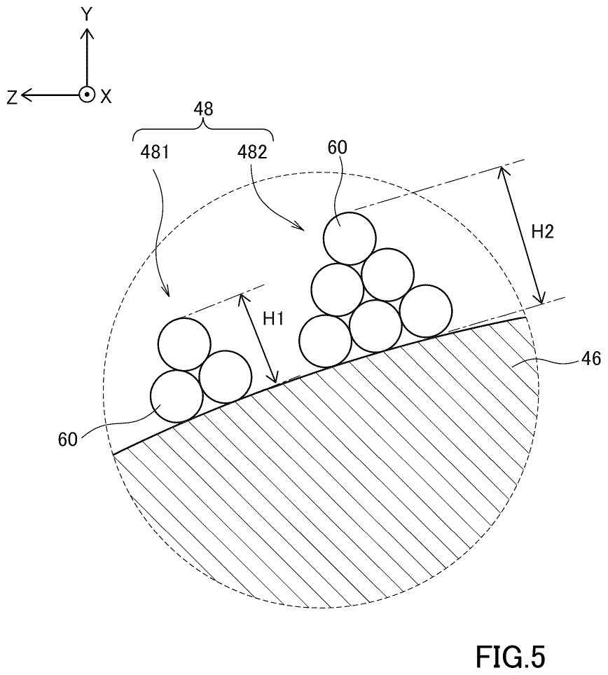

As illustrated in FIG. 5, the surface of the loop portion 46 is provided with a plurality of protruding portions 48. The protruding portion 48 is formed by particles 60 attached to the surface of the loop portion 46. The particle 60 is an example of an attachment substance. The number of the particles 60 included in the one protruding portion 48 may be one or two or more. The heights of the plurality of protruding portions 48 are not uniform. That is, the plurality of protruding portions 48 includes a first protruding portion 481 having a first height H1 and a second protruding portion 482 having a second height H2 different from the first height H1.

The protruding portion 48 may be provided on the entire surface of the loop portion 46 or may be partially provided. The two adjacent protruding portions 48 may be separated from each other or may be in contact with each other. The protruding portion 48 may be provided on the surface of the wide portion 461, may be provided on the surface of the distal end portion 462, may be provided on both the surface of the wide portion 461 and the surface of the distal end portion 462, or may be provided on a portion different from both the surface of the wide portion 461 and the surface of the distal end portion 462. The protruding portion 48 may be provided on the outer peripheral surface 46S1 of the wide portion 461, or may be provided on the outer peripheral surface 46S1 of the distal end portion 462. According to the present embodiment, the protruding portion 48 is provided on both the outer peripheral surface 46S1 of the wide portion 461 and the outer peripheral surface 46S1 of the distal end portion 462.

The material of the particle 60 may be, for example, metal, ceramic, diamond, or cermet.

Next, an example of a method for manufacturing the guide wire 100 will be described.

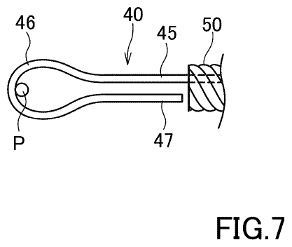

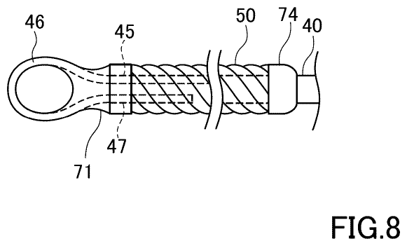

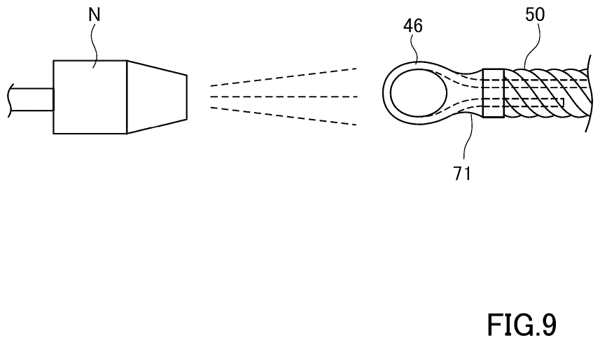

First, the core wire 40 is passed through the coil 50, and the distal end portion of the core wire 40 protrudes from the coil 50 toward the distal end side (S110). Subsequently, the distal end portion of the core wire 40 is bent into a loop shape using a pin P, thereby forming the loop portion 46 (S120, FIG. 7). After the bending process, the first small diameter portion 45 and the second small diameter portion 47 of the core wire 40 are inserted into the inside of the coil 50 and are joined to the coil 50 by the distal end side joining member 71. Subsequently, the core wire 40 is joined to the proximal end portion of the coil 50 by the proximal end side joining member 74 (S130, FIG. 8). After the joining, the loop portion 46 is subjected to a blasting process in which abrasive grains are blasted toward the loop portion 46 from a nozzle N provided in a blasting apparatus so that the particles 60 are attached to the surface of the loop portion 46 (S140, FIG. 9). Thus, the protruding portion 48 is formed. For example, the guide wire 100 according to the present embodiment is manufactured by the above-described process.

The process of attaching the attachment substance to the surface of the loop portion 46 may be a process other than the blasting process. The process of attaching the attachment substance may be, for example, a heat spraying process of spraying a material at least partially melted by heating, or a diamond electrodeposition process of fixing diamond abrasive grains to the surface of the target object by electroplating.

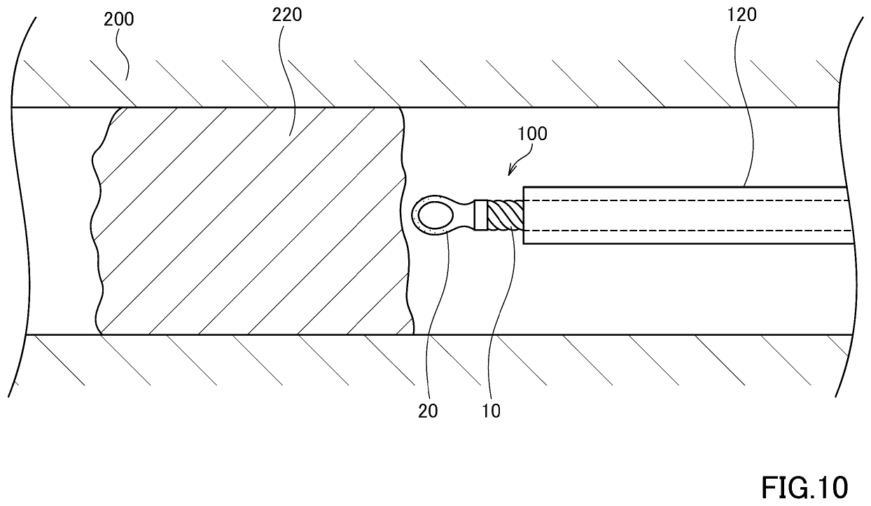

Next, an example of a treatment method using a catheter 120 including the guide wire 100 will be described. The professional inserts a preceding guide wire (not illustrated) into a blood vessel 200 and advances the preceding guide wire to the front of a lesion 220. The preceding guide wire is referred to as a workhorse guide wire, a first choice guide wire, or the like. Subsequently, the professional inserts the catheter 120 into the blood vessel 200 along the preceding guide wire. The professional advances the catheter 120 to the front of the lesion 220 in the blood vessel 200. Subsequently, the professional removes the preceding guide wire from the blood vessel 200. Then, the professional inserts the guide wire 100 into the catheter 120, which is inserted into the blood vessel 200, with the leading portion 20 at the head (FIG. 10). The professional advances the guide wire 100 to the front of the lesion 220 in the blood vessel 200. When the guide wire 100 is advanced, the guide wire 100 may be rotated around the center axis Ax or may not be rotated.

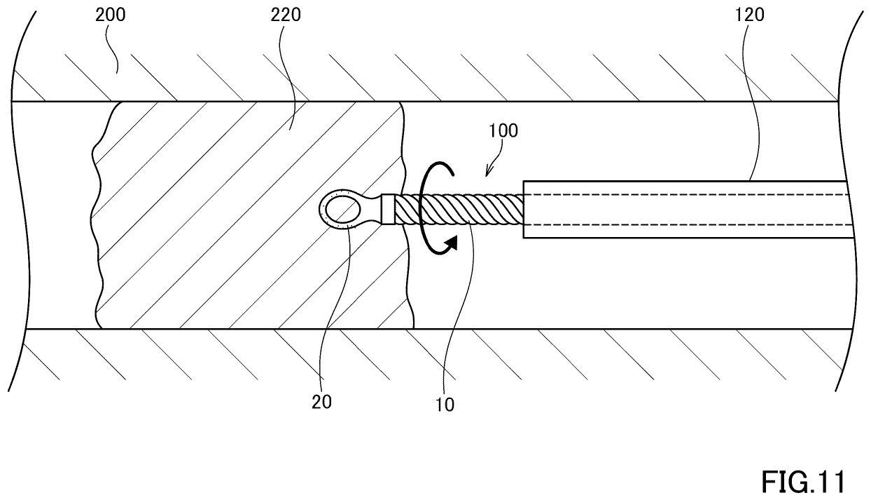

Next, the professional advances the leading portion 20 into the lesion 220 by advancing the guide wire 100 toward the distal end side while rotating the guide wire 100 (FIG. 11). When the professional grips the proximal end portion of the guide wire 100 and rotates the guide wire 100 around the center axis Ax, the leading portion 20 located at the distal end portion of the guide wire 100 also rotates around the center axis Ax. The leading portion 20 rotating in the lesion 220 excavates so as to cut the lesion 220. At this time, the protruding portion 48 is caught by the lesion 220 so that the guide wire 100 is prevented from slipping, and the lesion 220 is efficiently cut. This results in an improvement in the passing performance of the leading portion 20 with respect to the lesion 220. In particular, since the protruding portions 48 are provided on the wide portion 461 and the distal end portion 462 of the loop portion 46, which are likely to come into contact with the lesion 220, the excavation performance of the leading portion 20 with respect to the lesion 220 is improved. In addition, since the protruding portion 48 is provided on the outer peripheral surface 46S1 of the distal end portion 462, the protruding portion 48 is easily caught by the lesion 220. As a result, the leading portion 20 is prevented from slipping on the surface of the lesion 21 and rotating idly. The protruding portion 48 applies a microcrack to the lesion 220 so that the cut lesion 220 becomes a small fragment. This reduces the possibility that the lesion 220 clogs around the leading portion 20 and impedes the rotation of the leading portion 20.

As described above, the guide wire 100 according to the present embodiment includes the main body part 10 and the leading portion 20 that is connected to the distal end 16 of the main body part 10 and enters the lesion 220. The leading portion 20 includes the plurality of protruding portions 48 on a surface of the leading portion 20. With this configuration, the passing performance through the lesion 220 is improved.

According to the present embodiment, the leading portion 20 includes the loop portion 46 and the particle 60 attached to the surface of the loop portion 46. The protruding portion 48 includes the particle 60. With this configuration, the protruding portion 48 is easily formed at the leading portion 20.

According to the present embodiment, the leading portion 20 includes the wide portion 461 having an outer diameter larger than the maximum outer diameter D1 of the distal end 16 of the main body part 10 and the distal end portion 462 located closer to the distal end side than the wide portion 461. The protruding portion 48 is provided on the wide portion 461 and the distal end portion 462. With this configuration, the excavation performance of the lesion 220 is improved.

According to the present embodiment, the plurality of protruding portions 48 includes the first protruding portion 481 and the second protruding portion 482. The first protruding portion 481 has the first height H1. The second protruding portion 482 has the second height H2 different from the first height H1.

According to the present embodiment, the leading portion 20 includes the loop portion 46 having a loop shape, and the surface of the loop portion 46 is provided with the protruding portion 48. With this configuration, the guide wire 100 is efficiently manufactured.

In the method for manufacturing the guide wire 100 according to the present embodiment, the particles 60 are attached to the surface of the loop portion 46 provided at the leading portion 20, thereby forming the protruding portions 48 on the surface of the loop portion 46. With this configuration, the protruding portion 48 is easily formed in the leading portion 20.

### Second Embodiment

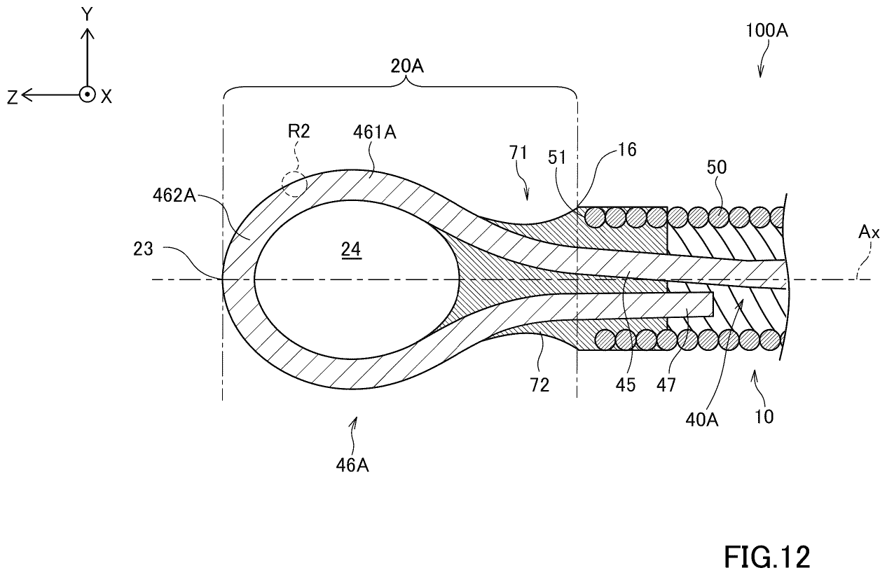

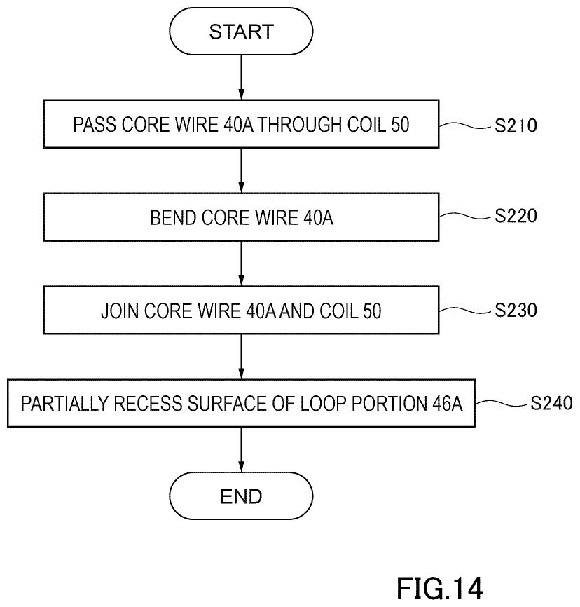

A second embodiment will be described with reference to FIGS. 12 to 14. In a guide wire 100A according to the present embodiment, the configuration of a leading portion 20A is different from that of the first embodiment. According to the present embodiment, the same components as those of the first embodiment are denoted by the same reference numerals, and description thereof will be omitted.

As in the first embodiment, the guide wire 100A according to the present embodiment includes the coil 50 and a core wire 40A. As in the first embodiment, the core wire 40A includes a portion from the large diameter portion 41 to the first small diameter portion 45, a loop portion 46A, and the second small diameter portion 47. As in the first embodiment, the loop portion 46A is a loop-shaped portion formed by a part of the core wire 40A. The detailed configuration of the loop portion 46A is the same as that of the first embodiment except that a recess portion 49 and a protruding portion 48A described below are provided instead of the protruding portion 48 according to the first embodiment.

The main body part 10 includes a portion of the core wire 40A from the large diameter portion 41 to the first small diameter portion 45, the second small diameter portion 47, the coil 50, a portion of the distal end side joining member 71 excluding the reinforcing portion 72, and the proximal end side joining member 74. The leading portion 20A includes the loop portion 46A formed by a part of the core wire 40A and the reinforcing portion 72. As in the first embodiment, the loop portion 46A includes a wide portion 461A having an outer diameter larger than the maximum outer diameter D1 of the distal end 16 of the main body part 10 and a distal end portion 462A located closer to the distal end side than the wide portion 461A.

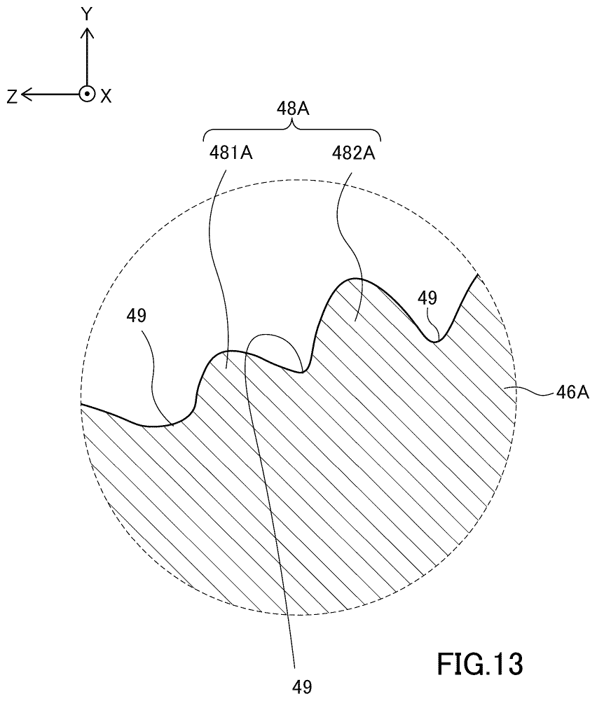

As illustrated in FIG. 13, the surface of the loop portion 46A is provided with a plurality of recess portions 49. On the surface of the loop portion 46A, the protruding portion 48A is a portion that is partitioned by the plurality of recess portions 49 and protrudes beyond the recess portion 49. The protruding portion 48A may be provided on the entire surface of the loop portion 46A, or may be partially provided. The protruding portion 48A may be provided on the surface of the wide portion 461A, may be provided on the surface of the distal end portion 462A, may be provided on both the surface of the wide portion 461A and the surface of the distal end portion 462A, or may be provided on a portion different from both the surface of the wide portion 461A and the surface of the distal end portion 462A. As in the first embodiment, the plurality of protruding portions 48A may include a first protruding portion 481A and a second protruding portion 482A having different heights. Next, an example of a method for manufacturing the guide wire 100A will be described.

First, the core wire 40A is passed through the coil 50, and the distal end portion of the core wire 40A protrudes from the coil 50 toward the distal end side (S210). Subsequently, the distal end portion of the core wire 40A is bent into a loop shape using the pin P, thereby forming the loop portion 46A (S220). After the bending process, the first small diameter portion 45 and the second small diameter portion 47 of the core wire 40A are inserted into the inside of the coil 50 and are joined to the coil 50 by the distal end side joining member 71. Subsequently, the core wire 40A is joined to the proximal end portion of the coil 50 by the proximal end side joining member 74 (S230). The process from steps S210 to S230 is the same as the process from steps S110 to S130 according to the first embodiment. After the completion of step S230, by partially recessing the surface of the loop portion 46A, the recess portion 49 is formed (S240). For example, the guide wire 100A according to the present embodiment is manufactured by the above-described processes.

The method for partially recessing the surface of the loop portion 46A may be, for example, a mechanical surface roughening process in which the surface of the loop portion 46A is partially ground by a blast process or polishing using sandpaper. The method may be a chemical surface roughening process in which the surface of the loop portion 46A is partially dissolved by an etching agent. The method may be an electrical surface roughening process in which the loop portion 46A is immersed in a solution for electrolytic polishing and an electric current is passed through the loop portion 46A as one electrode to partially dissolve the surface of the loop portion 46A.

As described above, the guide wire 100A according to the present embodiment includes the main body part 10 and the leading portion 20A, as in the first embodiment. The leading portion 20A includes the plurality of protruding portions 48A on a surface of the leading portion 20A. With this configuration, the passing performance of the leading portion 20A with respect to the lesion 220 is improved.

According to the present embodiment, the leading portion 20A includes the plurality of recess portions 49 that is recessed from the surface. The protruding portion 48A is a portion that is partitioned by the plurality of recess portions 49 and protrudes beyond the recess portion 49. In the method for manufacturing the guide wire 100A according to the present embodiment, the protruding portion 48A is formed by partially recessing the surface of the loop portion 46A provided at the leading portion 20A. With this configuration, the protruding portion 48A is easily formed in the leading portion 20A.

### Third Embodiment

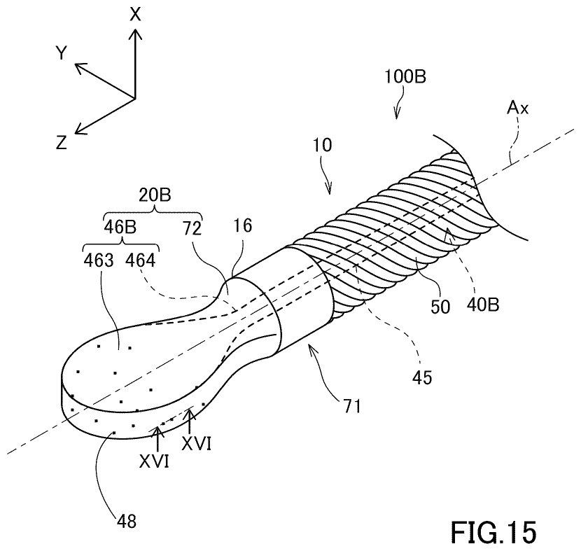

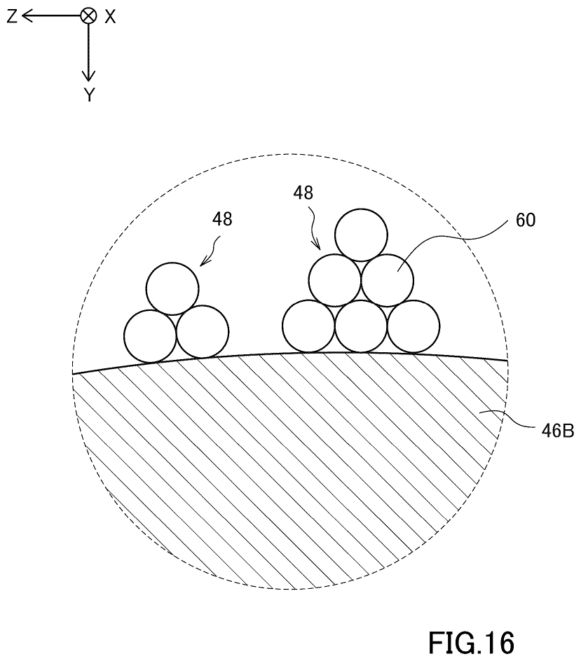

A third embodiment will be described with reference to FIGS. 15 and 16. In a guide wire 100B according to the present embodiment, the configuration of a leading portion 20B is different from that of the first embodiment. According to the present embodiment, the same components as those of the first embodiment are denoted by the same reference numerals, and description thereof will be omitted.

As in the first embodiment, the guide wire 100B according to the present embodiment includes the coil 50 and a core wire 40B. As in the first embodiment, the core wire 40B includes the portion from the large diameter portion 41 to the first small diameter portion 45. The core wire 40B further includes a paddle portion 46B. The paddle portion 46B is a portion that is continuous with the distal end of the first small diameter portion 45 and protrudes from the coil 50 toward the distal end side. The paddle portion 46B is an example of a base portion. The paddle portion 46B includes a plate portion 463 and a rod portion 464. The plate portion 463 is a plate-shaped portion having no through-hole. The outer shape of the plate portion 463 may be a circle, a partial circle, an ellipse, a rectangle, a parallelogram, a trapezoid, a rhombus, or the like, or may be a shape in which a part of these is distorted. According to the present embodiment, the plate portion 463 has a substantially-elliptical flat plate-shaped. The rod portion 464 is a linear portion that connects the plate portion 463 and the first small diameter portion 45. The paddle portion 46B may be formed, for example, by pressing a distal end portion of a wire rod that is a material of the core wire 40B. As illustrated in FIG. 16, the surface of the plate portion 463 is provided with the plurality of protruding portions 48. As in the first embodiment, the protruding portion 48 is formed by the particles 60 attached to the surface of the plate portion 463.

The main body part 10 includes a portion of the core wire 40B from the large diameter portion 41 to the first small diameter portion 45, the second small diameter portion 47, the coil 50, a portion of the distal end side joining member 71 excluding the reinforcing portion 72, and the proximal end side joining member 74. The leading portion 20B includes the paddle portion 46B formed by a part of the core wire 40B and the reinforcing portion 72.

As described above, the guide wire 100B according to the present embodiment includes the main body part 10 and the leading portion 20B, as in the first embodiment. The leading portion 20B includes the plurality of protruding portions 48 on a surface of the leading portion 20B. With this configuration, the passing performance of the leading portion 20B with respect to the lesion 220 is improved.

### Fourth Embodiment

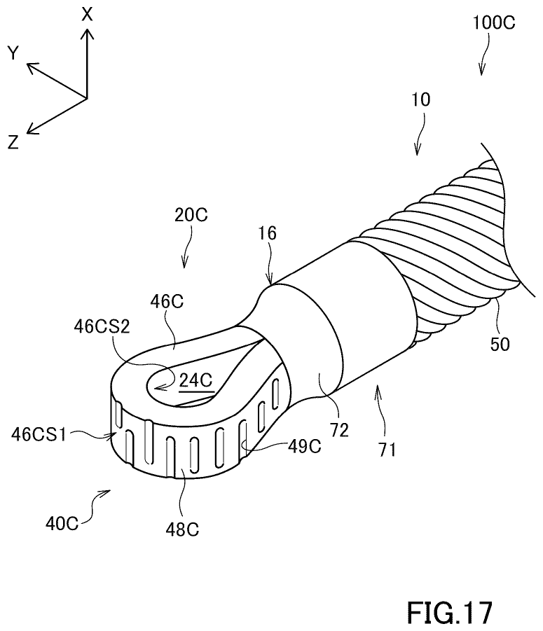

A fourth embodiment will be described with reference to FIG. 17. In a guide wire 100C according to the present embodiment, the configuration of a leading portion 20C is different from that of the first embodiment. According to the present embodiment, the same components as those of the first embodiment are denoted by the same reference numerals, and description thereof will be omitted.

As in the first embodiment, the guide wire 100C according to the present embodiment includes the coil 50 and a core wire 40C. The core wire 40C includes a loop portion 46C. The loop portion 46C is a loop-shaped portion formed by a part of the core wire 40C. The space inside the loop portion 46C is a through-hole 24C extending in the X-axis direction. The main body part 10 includes a portion of the core wire 40C excluding the loop portion 46C, the coil 50, a portion of the distal end side joining member 71 excluding the reinforcing portion 72, and the proximal end side joining member 74. The leading portion 20C includes the loop portion 46C and the reinforcing portion 72.

The surface of the loop portion 46C includes an inner peripheral surface 46CS2 facing the through-hole 24C and an outer peripheral surface 46CS1 provided back-to-back with the inner peripheral surface 46CS2. According to the present embodiment, the inner peripheral surface 46CS2 and the outer peripheral surface 46CS1 are parallel to each other.

The surface of the loop portion 46C is provided with a plurality of recess portions 49C and a plurality of protruding portions 48C. According to the present embodiment, the plurality of recess portions 49C and the plurality of protruding portions 48C are provided on the outer peripheral surface 46CS1. Each of the recess portions 49C extends in a direction substantially perpendicular to the circumferential direction of the loop portion 46C. The above-described recess portion 49C is formed by, for example, polishing the outer peripheral surface 46CS1 using sandpaper and, at this time, reciprocating the sandpaper in a direction perpendicular to the circumferential direction of the loop portion 46C. Polishing with sandpaper may be performed before the core wire 40C is bent to form the loop portion 46C, or may be performed after the core wire 40C is bent to form the loop portion 46C. Each of the protruding portions 48C is a portion that is partitioned by the two adjacent recess portions 49C and protrudes beyond the recess portion 49C.

As in the first embodiment, the guide wire 100C according to the present embodiment includes the main body part 10 and the leading portion 20C. The leading portion 20C includes the plurality of protruding portions 48C on a surface of the leading portion 20C. With this configuration, the passing performance of the leading portion 20C with respect to the lesion 220 is improved.

### Fifth Embodiment

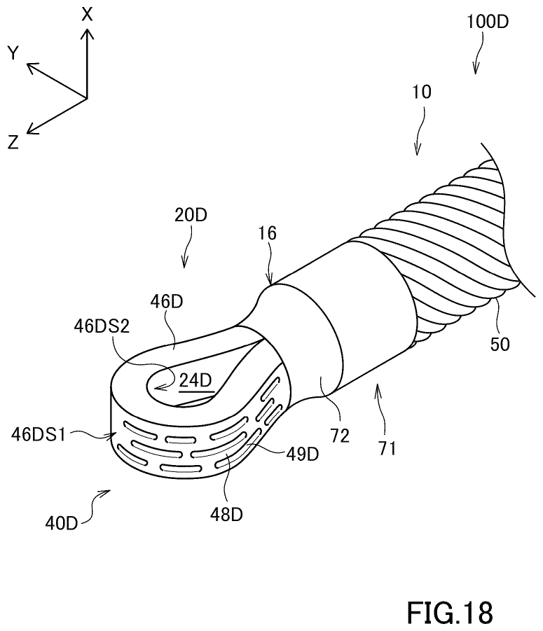

A fifth embodiment will be described with reference to FIG. 18. In a guide wire 100D according to the present embodiment, the configuration of a leading portion 20D is different from that of the first embodiment. According to the present embodiment, the same components as those of the first embodiment are denoted by the same reference numerals, and description thereof will be omitted.

As in the first embodiment, the guide wire 100D according to the present embodiment includes the coil 50 and a core wire 40D. The core wire 40D includes a loop portion 46D. The loop portion 46D is a loop-shaped portion formed by a part of the core wire 40D. The space inside the loop portion 46D is a through-hole 24D extending in the X-axis direction. The main body part 10 includes a portion of the core wire 40D excluding the loop portion 46D, the coil 50, a portion of the distal end side joining member 71 excluding the reinforcing portion 72, and the proximal end side joining member 74. The leading portion 20D includes the loop portion 46D and the reinforcing portion 72.

The surface of the loop portion 46D includes an inner peripheral surface 46DS2 facing a through-hole 24D and an outer peripheral surface 46DS1 provided back-to-back with the inner peripheral surface 46DS2. According to the present embodiment, the inner peripheral surface 46DS2 and the outer peripheral surface 46DS1 are parallel to each other.

The surface of the loop portion 46D is provided with a plurality of recess portions 49D and a plurality of protruding portions 48D. According to the present embodiment, the plurality of recess portions 49D and the plurality of protruding portions 48D are provided on the outer peripheral surface 46DS1. Each of the recess portions 49D extends along the circumferential direction of the loop portion 46D. The above-described recess portion 49D is formed by, for example, polishing the outer peripheral surface 46DS1 using sandpaper and, at that time, reciprocating the sandpaper along the circumferential direction of the loop portion 46D. Polishing with sandpaper may be performed before the core wire 40D is bent to form the loop portion 46D, or may be performed after the core wire 40D is bent to form the loop portion 46D. Each of the protruding portions 48D is a portion that is partitioned by the two adjacent recess portions 49D and protrudes beyond the recess portion 49D.

As in the first embodiment, the guide wire 100D according to the present embodiment includes the main body part 10 and the leading portion 20D. The leading portion 20D includes the plurality of protruding portions 48D on a surface of the leading portion 20D. With this configuration, the passing performance of the leading portion 20D with respect to the lesion 220 is improved.

### Modification

The technology disclosed herein is not limited to the above-described embodiments and can be modified into various forms without departing from the gist thereof, and for example, the following modifications are also possible.
(1) A part of the protruding portion may be provided at a portion of the leading portion different from the loop portion. A part of the protruding portion may be provided at the main body part.
(2) The loop portion may be formed as a member different from the core wire and connected to the main body part. The same applies to the paddle portion.
(3) The plate portion may not be flat plate-shaped. For example, the plate portion may have a plate shape having a recessed surface on its surface.
(4) The plate portion may include a recess portion that is recessed from the surface, and the portion that is partitioned by the recess portions and protrudes beyond the recess portions may be a protruding portion.
(5) The medical device may include a coated layer that covers the surface of the leading portion. In this case, the coated layer may have a thickness smaller than the height of the protruding portion.
(6) The leading portion may include no wide portion having an outer diameter larger than the outer diameter of the distal end of the main body part.
(7) The protruding portions may be regularly arranged or may be irregularly arranged.
(8) According to the above embodiment, the guide wire 100 for treating a lesion in a blood vessel has been described as an example. The technology disclosed herein is similarly applicable to medical devices in general for treating lesions in a living body lumen.

1. A medical device comprising:
a main body part; and
a leading portion that is connected to a distal end of the main body part and enters a lesion, wherein
the leading portion includes a protruding portion on a surface of the leading portion.

2. The medical device according to claim 1, wherein
the leading portion further comprises:
a base portion, and
an attachment substance that is attached to a surface of the base portion, and
the protruding portion includes the attachment substance.

3. The medical device according to claim 1, wherein
the leading portion includes a recess portion that is recessed from a surface, and
the protruding portion is a portion that is partitioned by the recess portion and protrudes beyond the recess portion.

4. The medical device according to claim 1, wherein
the protruding portion is a first protruding portion having a first height, and
the leading portion further includes a second protruding portion having a second height different from the first height.

5. The medical device according to claim 1, wherein
the leading portion includes a wide portion having an outer diameter larger than an outer diameter of the distal end of the main body part, and
the protruding portion is provided at the wide portion.

6. The medical device according to claim 1, wherein
the leading portion further comprises:
a wide portion having an outer diameter larger than an outer diameter of the distal end of the main body part, and
a distal end portion located closer to the distal end side than the wide portion, and
the protruding portion is provided at the distal end portion.

7. The medical device according to claim 1, wherein
the leading portion includes a loop portion having a loop shape, and
the protruding portion is provided on a surface of the loop portion.

8. The medical device according to claim 1, wherein
the leading portion includes a plate portion that has a plate shape and has no through-hole, and
the protruding portion is provided on a surface of the plate portion.

9. The medical device according to claim 1, wherein the protruding portion has a linear shape extending in a direction perpendicular to a circumferential direction of the loop portion.

10. The medical device according to claim 1, wherein the protruding portion has a linear shape extending in a circumferential direction of the loop portion.

11. A method for manufacturing a medical device comprising:
a main body part, and
a leading portion that is connected to a distal end of the main body part and enters a lesion,
the method comprising forming a protruding portion on a surface of a base portion provided at the leading portion by attaching an attachment substance to the surface of the base portion.

12. A method for manufacturing a medical device comprising a main body part, and
a leading portion that is connected to a distal end of the main body part and enters a lesion,
the method comprising forming a protruding portion on a surface of the leading portion by partially recessing the surface of the leading portion.
